(19) United States
(12) Patent Application Publication
(10) Pub. No.: US 2011/0311677 A1
(43) Pub. Date: Dec. 22, 2011

(54) NOVEL PACKAGING FOR LIQUID PRODUCTS

(76) Inventors: Emilie Bryckaert, Roubaix (FR); Jean-Pierre Chassard, Blaesheim (FR); Thomas Lotz, Lille (FR); Daniel Rebufello, Hem (FR)

(21) Appl. No.: 12/669,020

(22) PCT Filed: Jul. 16, 2008
(86) PCT No.: PCT/FR08/01042

§ 371 Date: Oct. 21, 2010

(30) Foreign Application Priority Data
Jul. 18, 2007 (FR) 07290898.1

(51) Int. Cl. B65D 6/00; C12N 1/16; A21D 8/04

(52) U.S. Cl. 426/19; 426/60; 220/660; 435/255.1

(57) ABSTRACT

The invention relates to a packaging for a liquid product containing yeast, that comprises at least one pervious material having an exchange surface S such that the ratio S/M is of at least 1.2, with S in cm² and M being the mass of the liquid product in g, and having an O₂ permeability coefficient CP of between 200 and 9000 cm³ /m² .24h.bar, and/or the O₂ permeability coefficient CP ranging from 800 to 45000 cm³ /m². 24h.bar. The invention also relates to the use of such a packaging for containing a liquid product containing yeast. The invention further relates to a preservation method and to the use of a liquid product containing yeast.

### FIELD OF THE INVENTION

The present invention relates to the field of packaging and the preservation of liquid products intended for foodstuffs, and more particularly relates to a package for the packaging of yeast-containing liquid products.

### TECHNOLOGICAL BACKGROUND

Yeast in aqueous suspension is a product particularly affected by its preservation conditions, in particular its environment (temperature, pH, CO₂/O₂ content, etc.), and particularly exposed to contamination. Therefore this is a particularly difficult product to package, thus requiring hygienic preservation conditions that simultaneously allow its microbiological quality, its performance, in particular in terms of fermentative capacity, and its organoleptic qualities to be maintained.

Moreover, the preservation of yeast in suspension in unsuitable conditions and variation in its preservation conditions have consequences with respect to its metabolism. Thus, an increase in fermentation and/or respiratory metabolism causes:

an overproduction of CO₂ with a reduction in pH by production of carbonates.

an increase in the consumption of reserve sugars by reduction in the trehalose content which leads to a reduction in its shelf life.

an overproduction of ethanol.

A loss of microbiological quality and performance, in particular of fermentative capacity and a degradation of its organoleptic qualities can then result.

Although its activity and reactivity ensure good performances in use, they constitute a specific drawback in the preservation of such a product. For its correct preservation, it is then desirable in practice to keep yeast in aqueous suspension at a low temperature, of the order of 4° C., and to provide specific degassing means, in particular for the release of the gases produced by yeast, in particular CO₂. Apertures forming vents have thus been described as specific degassing means, in patent EP792930B1 to the Applicant and in patent applications WO04048253A1 and WO0202428A1. It is also desirable to limit gaseous exchanges (oxygen from the ambient air) in particular in order to limit the development of contaminants.

These preservation requirements are thus incompatible with the constraints of the consumer market, where the act of purchasing by the consumer often leads to a break in the cold chain and where the packaging is handled frequently, shaken and turned over. Furthermore, the presence of a vent which is necessarily above the level of the liquid in particular makes handling by the general public difficult.

This is the reason why such a product is to date only marketed to the bakery trade.

The Applicant has already described in one of his previous patent applications WO04048253A1 that fresh yeast in aqueous suspension can be preserved in a package having a volume from 1 to 100 litres, which is kept in a cooler at a temperature of −2 to 12° C. Although this packaging constitutes a significant technological advance, the Applicant has noted that this solution still does not give full satisfaction, in particular in the case of yeasts having particularly active metabolisms and/or preservation temperatures above 5° C. and/or when the packs are shaken. In practice, this also often requires an additional vent to be provided. Long preservation over a period of 6 to 8 weeks is not required for this type of packaging inasmuch as the latter is directed to the bakery trade and cream yeast is intended for rapid consumption. This packaging is thus not intended for use by the general public, involving preservation for a relatively long period of time (typically 8 weeks) and preservation conditions often involving a break in the cold chain and frequent shaking of the packaging.

In patent application WO0202428A1, a packaging provided with a degassing vent was described, the volume of which is comprised between 0.1 and 100 litres. The packaging which is the subject of this application has the same drawbacks as those of the previous document. Again, such a packaging therefore does not constitute a suitable solution that can be exploited in order to respond to the constraints of the market for the general public.

Of course, packaging solutions have been described in the literature for packing food products such as fruits, vegetables and cheese, but the preservation of these products does not require a packaging which degasses CO₂ and/or limits the oxygen supply, whilst isolating a product in liquid form from its environment. On the other hand, water produced during the preservation of certain products often constitutes an unwanted product that it is desired to release to the outside of this type of packaging. Thus, this type of packaging is not likely to provide technical solutions that can be exploited for the packaging of yeast-containing liquid products.

Packaging solutions have also been described in the literature for packing gaseous drinks but the preservation of these products in this case requires them to be leaktight in order to contain the dissolved gas. Thus, such packaging is not likely to provide technical solutions that can be exploited for the packaging of yeast-containing liquid products.

The difficulties described above thus constitute an obstacle to the use of yeast in suspension. Moreover, currently only yeasts in solid form, called pressed or dry yeasts, in which these problems are manifested differently, are offered on the market for the general public. Yet, when stored in favourable conditions, yeast in suspension exhibits incontrovertible advantages, in particular through its simplified use, pre-portioned, as well as its good performance; making this moreover a product greatly valued by the bakery trade. Similarly, no liquid leaven product whether containing yeast in suspension or not, is currently for sale on the market for the general public, for the same reasons. Furthermore, a liquid product containing liquid leaven type yeast nevertheless allows ease of use and the obtaining of leavened bread's characteristics, i.e. the organoleptic contribution that is highly sought after by the consumer (a flavour characterized by a tart or acetic odour of the crumb), a specific texture of the bread and an extension of the preservation period. As the market for bread machines for the general public is undergoing a strong development, the need for products of this type suitable for consumer requirements is becoming increasingly urgent.

Thus there is a need for a packaging, in particular having a reduced volume, suitable for preserving and maintaining the qualities of yeast-containing liquid products. This packaging is more particularly necessary for the preservation of yeast-containing liquid products within the context of the market for the general public, where the act of purchasing the product by the consumer frequently involves breaking the cold chain or even an actual thermal shock.

### SUMMARY OF THE INVENTION

A subject of the present invention is a packaging of yeast-containing liquid product comprising at least one permeable material of which the exchange surface S is such that the S/M ratio is at least 1.2 with S expressed in cm² and M the mass of the liquid product expressed in g, the coefficient of permeability (CP) to O₂ of which is comprised between 200 and 9,000 cm³/m².24h.bar, and/or the coefficient of permeability (CP) to CO₂ of which is comprised between 800 to 4,5000 cm³/m².24h.bar.

A subject of the invention is also the use of such packaging to contain a yeast-containing liquid product.

The present invention moreover relates to a method for the preservation and use of a yeast-containing liquid product comprising the following steps:

packaging the yeast-containing liquid product in the packaging according to the invention;

preservation of said packaging of yeast-containing liquid product until its use at a preservation temperature equal to, or less than 15° C., preferably equal to or less than 10° C., then

optionally, exposure to a temperature variation which can be up to 35° C. for a duration which can be up to 4 hours, then

use of the yeast-containing liquid product.

### DETAILED DESCRIPTION OF EMBODIMENTS

Surprisingly, such packaging makes it possible to keep the yeast-containing liquid products with a swelling that is very acceptable commercially, that is to say imperceptible, i.e. the volume increase remains less than or equal to 50% of the initial volume in the case of a flexible packaging, even without any perceptible deformation of the package, even at a preservation temperature which can be up to 15° C. The pressure variation inside the packaging can go up to an increase of for example 50 mbar, even 100 mbar, similarly, in the case of a rigid packaging (of the tray type with lid for milk or cream intended for coffee).

In such preservation conditions it further allows, after 8 weeks:

maintenance of the microbiological quality, with a loss of biomass less than 20% loss in yeast dry matter, preferably less than or equal to 17%, even more preferably less than 5% in particular for liquid leaven; and/or

loss of total mass less than 20%, preferably less than 10% and even more preferably less than or equal to 5% by weight in particular for cream yeast; and/or

in cream yeast, limiting the development of contaminants to a commercially acceptable limit, in particular the development of lactic bacteria, coliforms and/or non-baking yeasts, in particular for baker's cream yeast; and/or

maintenance of the performance, in particular in terms of fermentative capacity with a loss of fermentative capacity of less than 50%, preferably less than 30%, and even more preferably less than 20% with respect to the initial value on normal dough.

Particularly beneficial is the way in which these advantages do not change when the packaging experiences a break in the cold chain. A simulated intentionally excessive thermal shock at 35° C. for 4 hours does not result in any notable deterioration of the product, neither in terms of microbiological qualities nor in terms of performance.

Such packaging finally makes it possible to offer the general public consumer a product having advantageous properties with a use-by-date suitable for a possibly long distribution chain, as in the case of supermarkets. On average, the packaging according to the invention makes it possible to offer conditions for the preservation of yeast-containing liquid products up to 6 weeks, even 8 weeks, advantageously in particular in the case of the preservation conditions of products for the general public, with possible breaks in the cold chain.

According to the present invention, by yeast-containing liquid product is meant a liquid suspension, typically an aqueous suspension, comprising yeast. This is generally fresh yeast or resuspended dried yeast. According to a preferred embodiment of the present invention, the yeast is a fresh yeast. Advantageously, said yeast comprises at the time of its packaging at least 10⁵ colony-forming units (CFU) of yeast per gram, preferably at least 10⁸ colony-forming units (CFU) of yeast per gram, and advantageously at least 10⁹ colony-forming units (CFU) of yeast per gram.

The yeast-containing liquid product preferably has a content of at least 0.03% of live yeast cells by dry matter weight, preferably at least 0.1%, and even more preferably at least 5% by yeast dry matter.

The packagings according to the invention are particularly appropriate for the preservation of yeasts used for their fermentation activity. These are in particular yeasts belonging to the Saccharomycetaceae family (classification from The Yeasts, a taxonomic study, Kurtzman C P and Fell C. W., 4^{th} Edition, Elsevier, 1998). The invention therefore mainly relates to baker's yeasts, but can also relate to oenological, distiller's and/or brewer's yeasts for which problems of preservation in liquid form arise.

Oenological, distiller's and/or brewer's yeasts are preferably chosen from the Saccharomyces genus, in particular S. bayanus, and S. cerevisiae, in particular the uvarum, calbergensis, and cerevisiae varieties, the Kluyveromyces genus, in particular K. thermotolerans, the Brettanomyces genus, in particular B. bruxellensis, the Torulaspora genus in particular T. delbrueckii, individually or in a mixture.

Brewer's yeast is preferably a yeast chosen from Saccharomyces cerevisiae, Saccharomyces chevalierii and Saccharomyces boulardii.

The yeast-containing product is liquid, i.e. it has a viscosity of less than 20,000 centipoises, preferably less than or equal to 1,000 centipoises measured at a temperature of approximately 10° C. using a standard viscosimeter, for example a J. P, Selecta ST2001 viscosimeter (L1=needle; speed=10 rpm up to a viscosity of 600 centipoises, speed =1.5 rpm above 600 centipoises) on a 500 ml sample. Baker's yeasts typically are not liquid products.

The yeast-containing liquid product has a density preferably comprised between 1.01 and 1.25 and preferably comprised between 1.05 and 1.15.

By liquid yeast product is meant in particular, according to the present invention, cream yeast, preferably baker's yeast and liquid leaven.

By cream yeast, preferably baker's yeast, is meant a liquid suspension, typically an aqueous suspension, of live yeast cells preferably of baker's yeast, said suspension having a preferable dry matter content of at least 12% by weight and generally comprised between 12 and 50% by weight (extended definition of cream yeast). Preferably, liquid cream yeast conforms to the definition of cream yeast in the strict sense, i.e. it has a dry matter content between 12 and 25% by weight, and even more preferably between 15 and 22% by weight. However, the present invention is also useful for cream yeasts, preferably baker's yeast, having a higher dry matter content, i.e. at least 25% by mass, such as in particular the baker's cream yeasts known as high-density, containing one or more osmotic agents, such as for example polyhydroxy compounds and salts for food use. Such high-density baker's yeasts, which can in particular have a dry matter content from 25 to 48% by mass, or also from 25 to 46% by mass, are known and are for example described in WO91/12315 and WO03/048342.

By liquid leaven according to the invention is meant a liquid suspension, typically an aqueous suspension, of live yeast cells, preferably baker's yeast, live lactic bacteria cells and flour. Preferably, the liquid leaven has a dry matter content comprised between 12 and 20% by weight, and more preferably comprised between 15 and 17% by weight.

Ready-to-use stable liquid leavens for breadmaking suitable for packaging according to the invention are in particular those described in the Applicant's previous patents EP0953288-B1 and WO2004/080187-A1.

Advantageously, the liquid leaven is obtained by utilizing a culture medium comprising at least one non-malted cereal flour and water, seeding by at least one preparation of heterofermentative lactic acid bacteria and at least one yeast preparation, preferably by the additional use of at least one malted cereal flour supplying amylases or any equivalent source of amylases and/or at least one seeding by a preparation of homofermentative lactic acid bacteria. During its packaging, it thus preferably comprises at least 10⁶ colony-forming units (CFU) of lactic acid bacteria per gram and at least 10⁴ colony-forming units (CFU) of yeast per gram, and even more preferably at least 10⁹ colony-forming units (CFU) of lactic acid bacteria per gram, at least 10⁶ colony-forming units (CFU) of yeast per gram, has a stable final pH comprised between 4 and 4.3 and a dry matter content between 13 and 20% and preferably contains from 15 to 30 g/kg of lactic acid and from 6 to 10 g/kg of acetic acid. Its production is described in more detail in EP0953288. The Applicant described in this patent that the liquid leaven must be preferably preserved between 0 and 4° C. so that its qualities and performances can be maintained for a shelf life of 6 to 8 weeks. The packaging according to the present invention henceforth allows the preservation of the liquid leaven at a higher temperature which can go up to 15° C., preferably comprised between 5 and 15° C. with good maintenance of its microbiological and organoleptic qualities and its performances for a shelf life from 6 to 8 weeks.

Another ready-to-use liquid leaven for breadmaking that is particularly advantageous according to the present invention comprises a flour-based culture medium containing at least one cereal flour and water, said medium being seeded and fermented by at least homofermentative lactic acid bacteria which can biotransform lactic acid and being seeded by at least one preparation of yeasts, preferably baker's yeasts. The ready-to-use liquid leaven for breadmaking preferably also comprises at least one malted cereal flour supplying amylases or any equivalent source of amylases. It thus comprises 10⁸ colony-forming units (CFU) of lactic acid bacteria per gram, of which 60% are homofermentative and can biotransform lactic acid, at least 10⁶ colony-forming units (CFU) of yeast per gram, has a final stable pH comprised between 3.8 and 4.5 and a dry matter content between 27 and 35%, contains at least 7 g of acetic acid, preferably from 15 to 30 g/kg of lactic acid and from 7 to 10 g/kg of acetic acid. Its production is described in detail in WP2004/080187-A1.

Preferably, yeasts used for the production of leaven are Saccharomyces chevalierii yeasts, and the homofermentative bacteria are those of the species Lactobacillus plantarum and/or casei, the heterofermentative strains are those of the species Lactobacillus brevis.

According to a particularly advantageous embodiment, the liquid yeast product, preferably fresh, in particular liquid cream yeast and liquid leaven, is stabilized by the addition of one or more food stabilizers. These stabilizers delay or avoid the settling of the yeast cells of the suspension. Due to its(their) presence in the suspension, the liquid product of fresh yeast, preferably cream yeast or liquid leaven, retains its homogeneity longer when it is preserved without shaking. From the different food stabilizers that can be used for stabilizing cream yeast, there can be mentioned gums, such as xanthan gum, and thermally- and/or chemically-modified starches, such as acetylated distarch adipate responding to the definition of modified starch E1422. Such stabilized cream yeasts are for example described in EP-A-0 792 930.

Preferably, the yeast strains contained in the liquid product have a fermentative capacity on normal dough comprised between 70 and 190 ml in two hours, preferably comprised between 80 and 160 ml in two hours and even more preferably less than or equal to 140 ml in 2 hours. The fermentative capacity is measured according to the test described in column 5 of patent U.S. Pat. No. 5,741,695 carried out using a Burrows and Harrison fermentation meter in a short breadmaking system which can be summarized as follows:

Test A₁ (Fresh Compressed Yeasts in Normal Dough):

A weight of compressed yeast corresponding to 160 mg of dry matter is added to 20 g of flour incubated at the temperature chosen for the measurement, this yeast being mixed with 15 ml of water containing 27 g of NaCl per litre and 4 g of (NH₄)₂SO₄ per litre; this is mixed for 40 seconds using a spatula, so as to obtain a dough that is placed in a water bath adjusted to 30° C.; thirteen minutes after the start of mixing, the vessel containing the dough is hermetically sealed; the total quantity of gas produced is measured after 60, then 120 minutes or several hours; this quantity is expressed in ml at 20° C. and under 760 mm of Hg. For all yeasts capable of giving in 120 minutes a gas emission equal to or greater than 150 ml of CO₂, the quantity of fermentable sugars contributed only by the flour is a limiting factor; consequently, the test is modified as follows: a weight of yeast corresponding to 106 mg of yeast dry matter is added instead of 160 mg, and the reading for the quantity of gas produced is conventionally multiplied by 1.5.

Test A₅ (Fresh Compressed Yeasts in Sweet Dough):

Test identical to test A₁, but 4 g of saccharose is added to the flour; the total quantity of gas produced is measured after 60 minutes and 120 minutes, or several hours.

Preferably, the yeast strains contained in the liquid product do not include strains having a very strong fermentative capacity in normal dough, i.e. those the fermentative capacity of which is greater than 190 ml in 2 hours at 20° C.

Advantageously, the yeast strains used are strains said to be adapted to the presence of a weak organic acid, in particular by a an adaptation process as described below. The yeast strains were multiplied in the laboratory by standard methods, with adaptation to the presence of weak organic acid(s) according to the teaching of U.S. Pat. No. 4,318,991, with the addition of 0.1 g to 10 g of short-chain aliphatic carboxylic acids, such as aliphatic carboxylic acids having 2, 3 or 4 carbon atoms, and/or their salts per litre of wort.

The yeast strains used can be so-called cold-sensitive strains for producing cream yeast or liquid leaven, as described in particular in patent EP00878996.

The yeast or leaven compositions can also contain additives or adjuvants for the purpose of improving breadmaking and/or maintaining the homogeneity of the suspension. These additives can be oxidants such as ascorbic acid, reducing agents such as L-cysteine, enzyme preparations having one or more enzymatic activities such as preparations of amylase, xylanase, lipase and/or phospholipidase, oxidase such as glucose oxidase. These additives can also be one or more osmotic agents, such as for example food polyhydroxy compounds and food salts.

The quantity of yeast-containing liquid product can vary, provided that the features of the packaging are still complied with. According to an embodiment, the latter is multi-dose. Preferably, the quantity of yeast-containing liquid product contained in the packaging corresponds to one dose per usage of product for producing a breadmaking product, i.e. a quantity of less than or equal to 100 g and more preferably between 20 and 80 g. The volume of the liquid product can be less than or equal to 0.5 litre. Advantageously, the volume of the yeast-containing product is less than or equal to 0.1 litre, in particular less than or equal to 0.08 litre. Advantageously, the volume of the packaging is less than or equal to 0.1 litre.

The invention thus provides a packaging for a yeast-containing liquid product, preferably fresh yeast-containing liquid product, comprising at least one permeable material the exchange surface S of which is such that the S/M ratio is at least 1.2, preferably 1.3 with S expressed in cm² and M the mass of the liquid product expressed in g, the coefficient of permeability (CP) to O₂ of which is comprised between 200 and 9,000 cm³/m².24h.bar, and/or the coefficient of permeability (CP) to CO₂ of which is comprised between 800 to 45,000 cm³/m².24h.bar.

Advantageously the coefficient of permeability to O₂ is comprised between 800 and 2,000 cm³/m².24h.bar.

Also advantageously, the coefficient of permeability to CO₂ is comprised between 2,400 and 8,000 cm³/m².24h.bar.

The coefficient of permeability is also called the gas transmission coefficient. It is measured according to standard ISO 14105-2:2003.

The permeable material is generally impermeable to liquid water and preferably to water vapour. It is also possible to combine materials, wherein one brings the O₂/CO₂ permeability properties and the other brings the properties of impermeability to liquid water and/or water vapour.

According to the invention, the S/M ratio denotes the ratio of the exchange surface S of the material expressed in cm² with respect to the mass M of the yeast-containing liquid product expressed in grams. Thus, the measurement of surface area is carried out excluding packaging welds. The term “exchange surface” denotes this surface area facing the liquid product, directly or indirectly via one or more further layers having a higher permeability. The exchange surface can be, but is not necessarily, in (direct or indirect) contact with the liquid product. In particular, there can be a “head space” in the packaging, either flexible or rigid. In a package constituted by permeable material, the exchange surface is constituted by the whole internal surface of the package, excluding welds, whether or not the filling factor is maximal.

The material having the coefficient of permeability can be internal, within the sandwich, or external.

Advantageously, the S/M ratio is a value that increases, the lower the O₂ and/or CO₂ coefficient of permeability. Thus, advantageously, the S/M ratio is at least 1.3, preferably 1.4, advantageously at least 3, preferably comprised between 4 and 12, even more preferably between 4.5 and 10.

This makes it possible to obtain a gas flow rate suitable for the quantity of liquid product and the available surface area.

A high coefficient of permeability makes it possible to have a low available surface area, therefore a low S/M ratio. This will preferably apply to solid containers of the tray type with a lid, in which the lid will comprise the permeable film.

Conversely, if the coefficient of permeability is low instead, the surface area of exchange is larger and the S/M ratio higher. This will apply preferably to the flexible packaging of the sachet type, in which substantially the whole of the film forming the packaging will comprise the permeable film.

According to an embodiment, the product (S/M)×CP to O₂ is comprised between 4,000 and 20,000, preferably between 5,000 and 10,000. According to another embodiment, the product (S/M)×CP to CO₂ is comprised between 15,000 and 80,000, preferably between 20,000 and 40,000.

Moreover, depending on the packaged yeast strain type or the composition of the liquid leaven and their respective activities, as well as the preservation temperature, the permeability and/or the S/M ratio can be varied.

According to the invention, the coefficients of permeability (CP) to O₂ and to CO₂ are defined as the transmission coefficients of oxygen and carbon dioxide respectively expressed in cm³ per m² per 24h per bar (cm³/m².24h.bar) and measured according to the standard ISO 15105-2:2003 annex B by a gas chromatography katharometric detection method with an injection valve and sample loop. Before measurement, the material is conditioned for 48 hours at 23° C. and at a gas humidity level of 0% RH. Measurement of the coefficient of permeability is carried out at a temperature of 23° C., with a gas humidity of 0% RH. The outer surface of the material is subjected to the test gases and the measurements are carried out on 3 samples of 50 cm². The test gas is constituted by a mixture of 50% oxygen and 50% carbon dioxide. The chromatographic detection is carried out using a Porapak® Q detector with a detector temperature of 140° C., a filament current of 200 mA after chromatograph calibration with gas measurement standards having a known oxygen and carbon dioxide content.

For further details on the measurement of low coefficients of permeability (CP) to O₂, (i.e. less than 5000 cm³/m².24h.bar), measurement is carried out according to standards ISO 15105-2:2003 annex A and ASTM D 3985-05 using a Systech 8000 device. Before measuring, the material is conditioned for 48 hours at 23° C. and a gas humidity level of 0%RH. Measurement of the coefficient of permeability is carried out at a temperature of 23° C., with a gas humidity of 0% RH. The outer surface of the material is subjected to the test gases and the measurements carried out with 21% oxygen on 3 samples of 0.5 dm². The stabilization time is 24 hours.

If the detection threshold of the device is reached, it is possible to reduce the O₂ content of the test gas and/or the measured surface in order to return to detection conditions. It is then only necessary to weight the result obtained by the reduction in content applied and/or the reduction in surface area applied.

For measuring a CO₂ coefficient of permeability only, it is also possible to apply the flame ionization detection method in gas phase chromatography with injection valve and sample loop according to standard ISO 15 105-2:2003 annex B.

The permeable material constituting all or part of the packaging according to the invention is a material generally having gas diffusion properties. In general it does not include the materials sought for the porosity and/or microporosity properties, i.e. those which are called porous or microporous because they contain open pores (opening). According to a preferred embodiment, the permeable material is not microporous, in particular is not a microporous material having a pore size of 0.05-5 gm, in particular 0.1-2 μm, with a pore density of 30% or more.

The Applicant found in a particularly unexpected fashion that the following packaging is particularly suitable for packaging a yeast-containing liquid product, preferably fresh yeast-containing liquid product, and in particular liquid cream yeast, preferably baker's yeast:

a packaging comprising at least one material the surface area of which is such that the S/M ratio is at least 3 and the coefficient of permeability to O₂ is comprised between 800 and 4,000 cm³/m².24h.bar. The coefficient of permeability to CO₂ is preferably comprised between 3,000 and 15,000 cm³/m².24h.bar. Particularly advantageously, such packaging is particularly suitable for packaging a liquid yeast having a standard dry matter content, comprised between 15 and 24% by weight.

according to a preferred embodiment, a packaging as above comprising at least one material the surface area of which is such that the S/M ratio is comprised between 4 and 10, and the coefficient of permeability to O₂ of which is comprised between 800 and 2,000 cm³/m².24h.bar, preferably between 1,000 and 2,000 cm³/m².24h.bar. Particularly advantageously, such packaging is particularly suitable for packaging a liquid yeast having a standard dry matter content, comprised between 15 and 24% by weight and offering the advantages of limiting any odours that may be perceived as unpleasant by the consumer such as the odours of alcohol and certain aromatic notes, in particular those of sulphur and phenol, allowing improved preservation in terms of stability and easier resuspension. The development of contaminants is moreover reduced with this packaging.

These above-mentioned packagings are suitable for an application in the form of packs, preferably packs containing less than 100 g, in particular between 20 and 80 g of liquid product containing fresh yeast

a packaging comprising at least one material the surface area of which is such that the S/M ratio is at least 6 and the coefficient of permeability to O₂ is comprised between 400 and 4,000 cm³/m².24h.bar, preferably 400 and 1,000 cm³/m².24h.bar.

a packaging comprising at least one material the surface area of which is such that the S/M ratio is at least 1.2, preferably 1.3 and the coefficient of permeability to O₂ of which is comprised between 1,000 and 8,000 cm³/m².24h.bar, preferably between 3,000 and 7,000 cm³/m².24h.bar, more preferably 4,000 and 6,500 cm³/m².24h.bar. The coefficient of permeability to CO2 is preferably comprised between 4,000 and 24,000 cm³/m².24h.bar. Particularly advantageously, such packaging is particularly suitable for the packaging of a liquid yeast having a higher dry matter content, i.e. at least 25% by mass, in particular baker's cream yeasts called high density (dry matter content comprised between 25 and 46% by mass) containing one or more osmotic agents, such as for example polyhydroxy food compounds and salts for food use.

This packaging immediately above is suitable for an implementation in the form of packs, preferably packs containing at least 100 g, in particular between 200 and 1,200 g of liquid product containing fresh yeast, more particularly approximately 500 g.

The packagings which are the subject of the invention have an improved preservation in particular with respect to a long break in the cold chain of the order of 2 to 8 hours.

According to a first embodiment, the packaging according to the invention is entirely flexible and/or deformable. This constitutes an undeniable advantage of the present invention, as the problem of CO₂ diffusion with the risk of deformation or even bursting of the packaging is at a maximum in this form. In fact, a flexible and/or deformable packaging lacks resistance, changes its shape during handling, thus exposing a larger surface area to contact with the yeast-containing liquid product, which can then suddenly change the diffusion properties of the packaging and the preservation conditions of the product. A pack having a flat shape also offers a large exchange surface with the ambient atmosphere and therefore with the air containing oxygen and maximises the risks of poor preservation. A packaging according to the invention, due to its coefficients of permeability and its S/M ratio, then allows good preservation of the yeast-containing liquid product, preferably fresh yeast-containing liquid product.

The packaging according to the invention is preferably in the form of a pack.

When it is in the form of a pack, the packaging can have a substantially rectangular, circular or ovoid geometrical shape, having an upper part, a lower part and at least one side part.

It can preferably comprise, in a preferred form, at least one bellows in the side part and/or a bellows in the lower part and/or a bellows in the upper part.

The material constituting the pack can be identical or different from that constituting the bellows.

Preferably, the presence of the bellows allows an increase in the volume of the pack comprised between 10 and 50% with respect to the volume without bellows.

By way of example according to the invention, a pack can be envisaged for a content of yeast-containing liquid product of approximately 500 g, the dimensions of which (excluding welds) are approximately 15 cm×23 cm. The invention therefore relates to these packs having dimensions (excluding welds) 12-18 cm×18-25 cm.

Also by way of example, a pack can be envisaged for a content of approximately 40 g of yeast-containing liquid product, the dimensions of which are approximately 7.5 cm×12.5 cm. The invention therefore relates to these packs having dimensions (excluding welds) 6-9 cm×11-14 cm.

According to a second embodiment, the packaging according to the invention is rigid on one part of its surface area exposed to contact with the yeast-containing liquid product, preferably fresh yeast-containing liquid product. The exchange surface defined by the material can for example be fixed above to form a flexible lid, in particular an opening lid. This embodiment corresponds to the presentation of a unit dose or a multi-dose packaging of cream yeast for example in order to form a pot with a lid. This embodiment corresponds for example to a pot having a cylindrical shape with an inside diameter of 5 to 15 cm and a height of 2 to 5 cm.

According to a preferred embodiment, the packaging comprises the permeable material over all of its exchange surface. According to a variant embodiment, the packaging, which is preferably flexible and/or deformable is constituted by two or more materials of different types, and optionally having different coefficients of permeability, i.e. the exchange surface comprises two or more parts having different surface areas. It is then possible to define an average coefficient of permeability as the mean surface of the coefficients of permeability of the different parts. By way of example for two surfaces S1 and S2 having two coefficients CP1 and CP2, the mean coefficient will be defined as (CP1 Si+CP2S2)/(S1+S2). Particularly advantageously, the material is chosen from the class of synthetic polymers, in particular polyolefins and PET and organic films.

Among the synthetic polymers, the most suitable materials are polyolefins, in particular polypropylene, preferably oriented, polyethylene, preferably uncoated, low-density PE (LDPE), linear or not, high-density PE (HDPE), linear or not, and polymers chosen from certain grades of PET polyester and/or in small thicknesses.

By organic films is meant according to the invention in particular biodegradable polymers. There can be mentioned as examples cellulose films, starch-based films, protein-based films, to which a plasticizer has optionally been added and synthetic polymers. These biodegradable materials are preferably used in complexes, in combination with a material conferring an impermeability to water and/or water vapour to the packaging.

The coefficient of permeability can be adjusted according to the nature of the polymer used (or of the mixture of polymers or of several layers of polymers if necessary) and the thickness of the polymer film, the diffusion being a function of film thickness, method parameters (for example Corona treatment or not). A standard thickness is comprised between 5 and 100 gm, typically approximately 10 to 50 gm.

Advantageously, the permeable material is in the form of film and/or complexed form.

The packaging according to the invention can comprise printing on its external face and/or on a face of an internal film; the coefficient of permeability is determined for the packaging in its final form, with printing (taking into account the entirety of the surface area and determining a mean coefficient).

According to a preferred embodiment, the material is complexed with other materials, the permeabilities to O₂ and CO₂ of which are greater than those of the material and the function of which is to bring additional qualities to the complex, such as for example to improve the behaviour of the packaging, in particular its rigidity, its suitability for machine handling, its touch, its opening ability, and to provide a more suitable medium for printing, in particular, varnishes.

The material can be arranged on any layer, i.e. on the internal surface in contact with the yeast-containing liquid product, the intermediate layer or the external layer.

The material can be combined with other materials by standard techniques of lamination, complexing, co-extrusion, acrylic resin coating, known to a person skilled in the art.

By way of preferred example support, there can be mentioned papers such as Kraft paper, other polyolefins such as polyethylene, polypropylene, or porous/microporous materials.

A particularly useful material according to the invention is constituted by a multi-layer complex of OPP/Kraft paper/PE, the OPP material being the material having the lowest coefficient of permeability.

According to an advantageous embodiment, the packaging is provided with means allowing it to be resealed after use and to use the product later. In particular, a resealable package can be provided. In this case, the lid contains a pressure-sensitive adhesive which is uncovered on first use by tearing along the weld bead. This type of package is known for example from patent WO-A-9719867 and the patent applications in the name of Soplaril citing this application. The packaging can also be equipped with a zipper strip.

Preferably, the packaging according to the invention does not comprise any vent.

A subject of the present invention is also the use of a packaging as defined previously for preserving a yeast-containing liquid product, preferably fresh yeast-containing liquid product. Such packaging is more particularly used at temperatures below 15° C., preferably comprised between 4° C. and 15° C. and allows good preservation for at least 4 weeks, preferably at least 6 weeks, and even more preferably 8 weeks or more.

In these preservation conditions, such packaging can moreover be used with a potential temperature variation which can reach 35° C. for a maximum period of 8 hours, and preferably for 4 hours, and even more preferably can reach 20° C. for a maximum period of 2 hours.

A subject of the invention is also a method for the preservation and use of yeast-containing liquid product, preferably fresh yeast-containing liquid product and preferably baker's yeast-containing liquid product comprising the following steps:

packaging of the yeast-containing liquid product in the packaging according to the invention, preferably 5 days or less after its production, then

preserving said packaging of yeast-containing liquid product until its use at a storage temperature equal to or below 15° C., preferably equal to or below 10° C., then

optionally, exposure to a temperature variation which can reach 35° C. for a period which can be up to 4 hours, preferably reaching 20° C. for a maximum period of 2 hours, then

use of the yeast-containing liquid product.

According to an embodiment of said method, said method comprises the following steps:

packaging the yeast-containing liquid product, preferably fresh yeast-containing liquid product, preferably 5 days or less after its production, then

preserving said packaging of yeast-containing liquid product during its transport at a temperature comprised between 0 and 15° C., preferably approximately 4° C., then

preserving at a temperature comprised between 0 and 15° C., preferably between 8 and 10° C. for at least 2 weeks, preferably 4 to 8 weeks, and even more preferably 4 to 6 weeks after packaging the yeast-containing product then

optionally, exposure to a temperature variation which can reach 35° C. for a period which can be up to 4 hours, preferably reaching 20° C. for a maximum period of 2 hours, then

use of the yeast-containing liquid product, preferably at the latest 8 weeks after packaging of the yeast-containing product.

Preferably, after exposure to a temperature variation which can reach 35° C. for a period up to 4 hours, preferably reaching 20° C. for a maximum period of 2 hours, the packaging of yeast-containing liquid product, preferably fresh yeast-containing liquid product is again preserved until use at a temperature comprised between 0 and 15° C., preferably between 6 and 10° C.

The liquid product containing baker's yeast is used for producing a breadmaking product. It can be directly in the flour of the breadmaking products, in a short or long breadmaking system. It is thus used for producing pizzas, French-style breads, pastries, etc.

The liquid product containing oenological yeast, baker's yeast and/or distiller's yeast can be used for seeding the fermentation and/or propagation medium.

The invention also allows a specific combination to be produced. In fact, consumers appreciate the availability of products in a pre-portioned form, for ease of preparation of the recipe. The invention thus offers the combination of a packaging according to the invention with one or more other cookery ingredients, in particular breadmaking improvers, a particular quantity of flour and other pre-portioned ingredients.

The following examples are provided to illustrate the invention and should in no way be considered as limiting the scope of the invention.

### EXAMPLES

Test 1: Monitoring of Different Packagings for Cream Yeast at 8-10° C. for 8 Weeks

1. Materials:

The preservation in different types of packagings was monitored over 8 weeks at a fixed temperature of 8 to 10° C.

All the packagings of cream yeast tested had the following features:

flexibility in the form of packs of dimensions (8.5 cm−0.8 cm corresponding to the weld dimension)×(13 cm−0.8 cm corresponding to the weld dimension)×2 faces i.e. one surface of 187.9 cm² excluding the welds

S/M ratio=4.7

baker's cream yeast stabilized at 19% DM dosed at 40 g per pack packaged 4 days after its production and preserved at 8-10° C. for 4 weeks.

By inner layer in multi-layer complexes is meant the layer exposed to contact with the cream yeast.

The following packagings were tested:

packaging 1: three-layer complex containing a leaktight aluminium foil, comparable to those used for the packaging of vermicelli-like dried yeast.

packaging 2: pack constituted by a polymer material having an air flow rate greater than 0.1 Nlitre/hour/cm² under a AP of 12 mbar.

packaging 3 according to the invention: pack constituted by a material in a bi-layer complex, constituted by an outer layer made of Kraft paper 45 g/m² bonded to an inner polypropylene (PP cast) layer of thickness 25 μm; coefficient of permeability to O₂ of 380 cm³/m².24h.bar.

packaging 4 according to the invention: pack constituted by a polyethylene PE material, thickness 40 μm; coefficient of permeability to O₂ of 3,500 cm³/m².24h.bar; coefficient of permeability to CO₂ of 25,400 cm³/m².24h.bar.

packaging 5 according to the invention: pack entirely constituted by a bi-layer complex constituted by an outer layer of Kraft paper 60 g/m² coated with an inner layer of polyethylene PE 20 g/m²; coefficient of permeability to O₂ of 8,400 cm³/m².24h.bar; coefficient of permeability to CO₂ of 45,000 cm³/m².24h.bar.

packaging 6 according to the invention: pack entirely constituted by a bi-layer complex constituted by an outer layer of coextruded oriented polypropylene OPP having a thickness of 20 μm bonded to an inner layer of linear polyethylene LDPE of thickness 20 μm; coefficient of permeability to O₂ of 1,570 cm³/m².24h.bar; coefficient of permeability to CO₂ of 7,600 cm³/m².24h.bar.

packaging 7 according to the invention: pack entirely constituted by a multilayer complex constituted by an outer layer of oriented polypropylene OPP having a thickness of 20 μm bonded to an intermediate layer of Kraft paper 22 g/m² coated with an inner layer of polyethylene PE 6 g/m2; coefficient of permeability to O₂ of 1,600 cm³/m².24h.bar; coefficient of permeability to CO₂ of 5,400 cm³/m².24h.bar.

packaging 8 according to the invention: pack entirely constituted by a multilayer complex constituted by an outer layer of oriented polypropylene OPP having a thickness of 20 gm bonded to an intermediate layer of Kraft paper 45 g/m2 coated with an inner layer of polyethylene PE 15 g/m²; coefficient of permeability to O₂ of 1,630 cm3/m2.24h.bar.

packaging 9 according to the invention: pack entirely constituted by a multilayer complex constituted by an outer layer of oriented polypropylene OPP having a thickness of 20 gm bonded to an intermediate layer of Kraft paper 40 g/m² coated with an inner layer of polyethylene LDPE 20 μm; coefficient of permeability to O₂ of 1,350 cm3/m2.24h.bar.

The quality of the cream yeast was determined after 8 weeks of preservation at 8-10° C. via observation of the appearance of the cream, its microbiology and its weight.

The performances correspond to the measurements of fermentative capacity on normal and sugared dough (breadmaking application in French-type bread and brioche), the development of dry matter content levels, ethanol and trehalose assays.

The other parameters of use correspond to assessment of swelling, odour, settling and the ability of the cream to be emptied from its packaging.

2. Results:

| | Quality | | Other | |
|---|---|---|---|---|
| | of the | | parameters | |
| Packaging | cream | Performance | of use | comments |
| 1 control | ND | ND | ∘ (bursting) | |
| 2 control | o (dried | ND | ∘ (emptying | |
| | out) | | very difficult) | |
| 3 invention | x | x | ∘ (slight | |
| | | | swelling) | |
| 4 invention | x | x | x | alcohol odours |
| | | | | (notes of phenol) |
| 5 invention | x | x | ∘ (slight | alcohol odours |
| | | | settling) | (notes of |
| | | | | phenol) |
| | | | | no swelling |
| 6 invention | x | x | x | |
| 7 invention | x | x | x | |
| 8 invention | x | x | x | |
| 9 invention | x | x | x | |

NA = not determinable
∘ = unsatisfactory results
x = satisfactory results

With respect to packaging 1, it should be noted that bursting of the packs was also observed at preservation temperatures of 4° C. and 20° C. The internal pressure caused by the accumulation of the gases produced by yeast (CO₂ and ethanol) causes the welds of the packs to break in less than 2 weeks.

With respect to packaging 2, a loss of 80% of the initial mass occurred after 2 weeks of preservation. The loss of biomass was such that no measurement of performance was possible.

With respect to packaging 3, a slight swelling was observed.

The packagings according to the invention showed acceptable performances in terms of fermentative capacity tested on breadmaking dough. The loss of fermentative capacity after 6 weeks with the packagings according to the invention is less than or equal to 50% in normal dough, and remains acceptable for use in sweet dough. Moreover, whatever breadmaking system is used, the liquid products preserved in the packagings according to the invention gave very satisfactory breadmaking products.

3. Conclusion:

Packagings 1 and 2 cannot be preserved in market conditions for the general public, in particular at 8-10° C. The packagings according to the invention are the only ones allowing preservation of the qualities during the preservation phase at 8° -10° C., i.e. according to the conditions of the market for the general public without breaking the cold chain. After 8 weeks of preservation without breaking the cold chain such packagings allow the consumer to be provided with the qualities of cream yeast in a very acceptable fashion. Packagings 6 to 9 are those giving the best results in particular as they limit settling and allow easy emptying of the cream during use. Moreover, no odour of alcohol is perceived by the consumer with these packagings. Packagings 6, 8 and 9 are those which moreover can be better handled by machine (strength of the welds).

Test 2: Variation of the S/M ratio and Storage Conditions

In the conditions of test 1 above, the S/M ratio was varied for 3 packagings; 3, 4 and 9. The preservation conditions were also varied.

The results are shown in the table below, a slight swelling or medium swelling also being acceptable, even if avoiding such swelling is preferable.

| | | Swelling | | |
|---|---|---|---|---|
| S/M | Type of complex | 3 wks at 4° C. | 10° C. (*) | 20° C. (**) |
| 1.61 | 4 | no | medium | medium |
| 3.22 | 4 | no | no | no |
| 3.22 | 9 | no | no | medium |
| 4.7 | 4 | no | no | no |
| 4.7 | 9 | no | no | no |
| 9.9 | 4 | no | no | no |
| 9.9 | 9 | no | no | no |
| 9.9 | 3 | no | no | medium |

(*): Thermal treatment applied: 2 wks at 4° C. + 1 wk at 10° C. OR 1 wk at 4° C. + 2 wks at 10° C.
(**): Thermal treatment applied: 1 wk at 4° C. + 2 wks at 20° C. OR 1 wk at 4° C. + 1 wk at 10° C. + 1 wk at 20° C.

Test 3: Monitoring Of Different Packagings of Cream Yeast at 8-10° C. with Simulation of the Market for The General Public for 8 Weeks

A comparison was carried out between the examples according to the invention 4 and 9. The S/M ratio was still 4.7.

The preservation conditions after packaging of the baker's cream yeast were the following:

8 weeks at 8-10° C. with application of a thermal shock of temperature 35° C. for 4 hours at 4 weeks. The analysis was thus carried out 8 weeks after the packaging of the cream yeast.

The following parameters were then assessed:

the loss in dry matter (as a percentage with respect to the initial value during packaging).

the alcohol content (ethanol content in g/l).

the trehalose content (content as a percentage of trehalose dry matter).

the fermentative capacity (application in breadmaking to French-type bread and brioche).

the other parameters of use (assessment of the swelling, odour, settling and ability to empty the cream from its packaging).

Packaging 4 did not show swelling of the pack, but an alcohol odour was noted (an ethanol content less than that of the packaging 9 being noted in the liquid product; difference of approximately 32%), a greater loss of biomass than that of packaging 9, a greater loss of trehalose (trehalose loss greater in packaging 4 than in packaging 9; difference of approximately 15%), and a greater contamination (contaminants of non-baker's type yeast and/or bacteria). The stability of the fermentative performances was slightly higher with packaging 9 compared with packaging 4.

These results show that these two packagings are suitable for use by the general public, the packaging 9 nevertheless giving better results than the packaging 4.

1. A packaging for a yeast-containing liquid product, comprising at least one permeable material the exchange surface S of which is such that the S/M ratio is at least 1.2, with S expressed in cm² and M the mass of the liquid product expressed in g, the coefficient of permeability (CP) to O₂ of which is comprised between 200 and 9,000 cm³/m².24h.bar, and/or the coefficient of permeability (CP) to CO2 of which is comprised between 800 to 45,000 cm³/m².24h.bar.

2. The packaging of claim 1, wherein the coefficient of permeability (CP) to O₂ is comprised between 800 and 2,000 cm³/m².24h.bar.

3. The packaging of claim 1, wherein the coefficient of permeability (CP) to CO2 is comprised between 2,400 and 8,000 cm³/m².24h.bar.

4. The packaging claim 1, wherein the S/M ratio is at least 1.3.

5. The packaging of claim 1, wherein the product (S/M)×CP to O₂ is comprised between 4,000 and 20,000.

6. The packaging of claim 1, wherein the product (S/M)×CP to CO2 is comprised between 15,000 and 80,000.

7. The packaging of claim 1, wherein the S/M ratio is at least 3 and the coefficient of permeability to O₂ is comprised between 800 and 4,000 cm³/m².24h.bar and the coefficient of permeability to CO2 is comprised between 3,000 and 15,000 cm³/m².24h.bar.

8. The packaging of claim 1, characterized in that it is flexible and/or deformable.

9. The packaging of claim 8, characterized in that it is presented in the form of a pack having a substantially rectangular, circular or ovoid geometrical shape, having an upper part, a lower part and at least one side part, each part comprising, individually or together, a bellows.

10. The packaging of claim 8, characterized in that it has a volume less than or equal to 0.5 litre.

11. The packaging according to one of claims 1, characterized in that it has a volume less than or equal to 0.1 litre.

12. The packaging of claim 1, characterized in that it is rigid and comprises a flexible lid fixed on top.

13. The packaging of claim 12, characterized in that it has a cylindrical shape having an inside diameter of 5 to 15 cm and a height of 2 to 5 cm.

14. The packaging of claim 1, wherein the packaging comprises said permeable material over all of its exchange surface.

15. The packaging of claim 1, wherein the permeable material is in the form of film and/or in a complexed form.

16. The packaging of claim 1, wherein the permeable material has a thickness comprised between 5 and 100 μm.

17. The packaging of claim 1, wherein the permeable material is chosen selected from the group consisting of synthetic polymers and organic films.

18. The packaging of claim 1, wherein the permeable material is not microporous, does not include a vent and is impermeable to liquid water and preferably to water vapour.

19. The use of the packaging of claim 1 for containing a yeast-containing liquid product.

20. The use of claim 19 wherein the yeast-containing liquid product has a content of at least 0.03% by weight of dry matter of live yeast cells.

21. The use of a packaging of claim 19, wherein the yeast-containing liquid product is selected from the group consisting of cream yeast and liquid leaven.

22. A method for the preservation and use of a yeast-containing liquid product, comprising the following steps:
packaging the yeast-containing liquid product in the packaging of claim 1;
preserving said packaging of yeast-containing liquid product until its use at a storage temperature equal to or below 15° C., then
optionally, exposure to a temperature variation which can be up to 35° C. for a duration which can be up to 4 hours, then
use of the yeast-containing liquid product.

23. The method of claim 22, wherein the yeast-containing liquid product is characterized according to claim 20.

24. The method of claim 22, comprising the following steps:
packaging the yeast-containing liquid product in the packaging of claims 1;
preserving said packaging of yeast-containing liquid product during its transport at a temperature comprised between 0 and 15° C., then
preserving at a temperature comprised between 0 and 15° C., for at least 4 weeks, after packaging of the yeast-containing product, then
optionally, exposure to a temperature variation which can be up to 35° C. for a duration which can be up to 4 hours, then
use of the yeast-containing liquid product.

25. The method of claim 22, comprising the following steps:
exposure to a temperature variation which can be up to 35° C. for a duration which can be up to 4 hours, then
preserving until its use at a temperature comprised between 0 and 15° C.

26. The method of claim 22, in which wherein the yeast-containing product is used to produce a breadmaking product.
